(19) United States
(12) Patent Application Publication
(10) Pub. No.: US 2010/0137694 A1
(43) Pub. Date: Jun. 3, 2010

(54) RADIO TRANSPARENT SENSOR IMPLANT PACKAGE

(75) Inventors: Pedro P. Irazoqui, Lafayette, IN (US); Arthur Chlebowski, Granger, IN (US); Casey Ellison, Lafayette, IN (US); William J. Chappell, West Lafayette, IN (US)

Correspondence Address: Vern Maine & Associates, 100 MAIN STREET, P O BOX 3445, NASHUA, NH, 03061-3445, US

(73) Assignee: Purdue Research Foundation, West Lafayette, IN (US)

(21) Appl. No.: 12/629,420

(22) Filed: Dec. 2, 2009

Related U.S. Application Data
(60) Provisional application No. 61/119,151, filed on Dec. 2, 2008.

(51) Int. Cl. A61B 5/07; A61B 3/16; H01P 11/00

(52) U.S. Cl. 600/302; 600/398; 29/600

(57) ABSTRACT

A system is provided for the packaging of wireless electronics in a mammalian body, the system comprising: a package configured in a shape suitable for implantation in that body; the package being configured from a biocompatible material; and that biocompatible material having a high degree of radio wave transparency.

### RELATED APPLICATIONS

This application claims the benefit of U.S. Provisional Application No. 61/119,151, filed Dec. 2, 2008. This application is herein incorporated by reference in its entirety for all purposes.

### FIELD OF THE INVENTION

The invention relates to sensor packages, and more particularly, to a sensor package having high radio transparency.

### BACKGROUND OF THE INVENTION

Novel packaging capabilities targeted at reducing the size and increasing reliability are required for the advancement of biomedical implants. Biocompatible metals, used for chronic implants, and epoxies, used for short-term applications, are the two most common materials used in the packaging of biological implants.

Current biocompatible packaging materials all have limitations for implantable use. Epoxies and metals are the most commonly used materials because of their availability and ease of use. The major drawback of epoxy is that it can only be used for acute research due to the quick degradation of its polymer backbone which exposes the implant to the surrounding tissue. Chronic implantation requires an alternative material with long term reliability. Biocompatible metals which are commonly used for encasing implants hinder wireless telemetry because the casing creates a faraday cage. Wireless capabilities are compulsory because of aesthetics, comfort, and most importantly, reduced risk of infection. A novel set of packaging materials that enables wireless transmission of data and can be chronically implanted could provide tremendous advantages to existing and future biomedical technology.

Current microelectronic techniques are advancing everyday communication devices. From cell phones to radios, microelectronics offers the ability to make these portable devices more powerful, reliable, and smaller. These technological advancements need to be transitioned into biomedical devices. In some non-biomedical applications Low Temperature Co-fired Ceramics (LTCC) is used to achieve compact size and improved performance. These LTCC materials allow for the building of components, resistors, capacitors, and inductors, that when assembled on successive layers, and then combining those layers together, create a single product. Therefore smaller packages are created, removing commonly used off the shelf components. LTCC is not the only material that enables these new, smaller implantable devices. Silicon and Liquid Crystal Polymer (LCP) each enable the formation of conductive traces created on its surface. Utilization of such materials in implantable devices, could make these devices smaller and more efficient, minimizing trauma during surgery and producing better post-operative performance. Methods for such utilization have not been employed.

Among the challenges associated with utilizing such technologies in implantable devices, is the requirement that packages comprising such new materials will be required to pass standard biocompatibility testing, such as long-term implant and cytotoxicity testing.

What is needed, therefore, are techniques for constructing an biocompatible, radio transparent package.

### SUMMARY OF THE INVENTION

One embodiment of the present invention provides a system for the packaging of wireless electronics and sensors in a mammalian body, the system comprising: a package configured in a shape suitable for implantation in the body; the package being configured from a biocompatible material; and the biocompatible material having a high degree of radio wave transparency.

Another embodiment of the present invention provides such a system wherein the biocompatible material is selected from the group of biocompatible materials consisting of liquid crystal polymer, poly(methyl methacrylate), low temperature co-fired ceramic, anisotropic conductive adhesive, silicon and combinations of thereof.

A further embodiment of the present invention provides such a system 1 wherein the package is provided with a plurality of layers of the biocompatible material, the layers being applied in a pattern, the pattern defines a structure in the shape suitable for implementation in the body.

Still another embodiment of the present invention provides such a system wherein the high degree of radio wave transparency is a degree of transparency that allows signals transmitted from an antenna disposed within the package to be transmitted to a receiver disposed externally to the body at a distance of about approximately 2 meters.

A still further embodiment of the present invention provides such a system wherein the high degree of radio wave transparency comprises a loss of not greater than 50 dB from an antenna disposed within a mammalian body.

Yet another embodiment of the present invention provides such a system wherein the high degree of radio wave transparency comprises having not greater than 50 dB of loss from an antenna disposed within the package.

A yet further embodiment of the present invention provides such a system further comprising a biocompatible coating.

Even another embodiment of the present invention provides such a system wherein the biocompatible coating comprises parylene.

One embodiment of the present invention provides a method for the manufacture of an implantable electronics package, the method comprising: Placing a first biocompatible material sheet on a first mold plate; Patterning an antenna and metal contacts upon a top surface of the first biocompatible material sheet; Disposing an electronics package upon the metal contacts; Draping a second sheet of biocompatible material over the first biocompatible material sheet and the electronics package; and Compressing the second sheet of biocompatible material against the top surface with a second mold plate having a recess for receiving the electronics package.

Another embodiment of the present invention provides such a method wherein the biocompatible material is a biocompatible selected from the group of materials consisting of liquid crystal polymer, poly(methyl methacrylate), low temperature co-fired ceramic, anisotropic conductive adhesive, silicon and combinations of thereof.

A further embodiment of the present invention provides such a method further comprising: creating a fenestration cut into the first biocompatible material sheet, thereby allowing internal components to interact with an environment external to the package.

Still another embodiment of the present invention provides such a method wherein the creating the fenestration comprises deep reactive ion etching the first biocompatible material sheet forming the fenestration.

A still further embodiment of the present invention provides such a method further comprising hermetically sealing the first biocompatible material sheet proximate to the fenestration with Anisotropic conductive adhesive to least one component of the electronics package, the component being disposed proximate to the fenestration, such that at least a portion of the component is exposed to the environment external to the implantable package.

One embodiment of the present invention provides a method for creation of a hermetic seal in an implantable package, the method comprising: Placing a first biocompatible material sheet on a first mold plate; Patterning an antenna and metal contacts upon a top surface of the first biocompatible material sheet; Disposing an electronics package upon the metal contacts; Draping a second sheet of biocompatible material over the first biocompatible material sheet and the electronics package; and laser sealing the second sheet of biocompatible material to the top surface with a second mold plate having a recess for receiving the electronics package.

Another embodiment of the present invention provides such a method further comprising: creating a fenestration cut into the first biocompatible material sheet, thereby allowing internal components to interact with an environment external to the package.

A further embodiment of the present invention provides such a method wherein the creating the fenestration comprises deep reactive ion etching the first biocompatible material sheet forming the fenestration.

Still another embodiment of the present invention provides such a method further comprising hermetically sealing the fenestration with Anisotropic conductive adhesive to least one component of the electronics package, the component being disposed proximate to the fenestration, such that the component is exposed to the environment external to the implantable package.

A still further embodiment of the present invention provides such a method wherein the first and second biocompatible material sheets are each comprised of a biocompatible material selected from the group of biocompatible materials consisting of liquid crystal polymer, poly(methyl methacrylate), low temperature co-fired ceramic, anisotropic conductive adhesive, silicon and combinations of thereof.

The features and advantages described herein are not all-inclusive and, in particular, many additional features and advantages will be apparent to one of ordinary skill in the art in view of the drawings, specification, and claims. Moreover, it should be noted that the language used in the specification has been principally selected for readability and instructional purposes, and not to limit the scope of the inventive subject matter.

### BRIEF DESCRIPTION OF THE DRAWINGS

FIG. 1A is a perspective drawing illustrating deposition of metal pads and antenna on a biocompatible sheet in accordance with one embodiment of the present invention.

FIG. 1B is a perspective drawing illustrating disposition of a second biocompatible sheet atop electronics and metallic pads and antenna and a first biocompatible sheet in accordance with one embodiment of the present.

FIG. 1C is a perspective drawing illustrating disposition of a second biocompatible sheet atop electronics and metallic pads and antenna and a first biocompatible sheet having electronic components disposed on the first sheet in accordance with one embodiment of the present.

FIG. 1D is a perspective drawing illustrating disposition of another biocompatible sheet atop said package in accordance with one embodiment of the present.

FIG. 1E is a perspective drawing illustrating an implantable package in accordance with one embodiment of the present.

FIG. 1F is a perspective drawing illustrating laser sealing implantable package in accordance with one embodiment of the present.

FIG. 2 is a perspective drawing illustrating an implantable electronic device package having a liquid crystal polymer body configured in accordance with one embodiment of the present invention.

FIG. 3A is an elevation drawing illustrating deposition of a metalized layer on a biocompatible sheet in accordance with one embodiment of the present invention.

FIG. 3B is an elevation drawing illustrating disposition of a Photoresist layer on masking the metalized layer in accordance with one embodiment of the present.

FIG. 3C is an elevation drawing illustrating etching of the metalized layer and removal of the Photoresist mask in accordance with one embodiment of the present.

FIG. 3D is an elevation drawing illustrating disposition of a second Photoresist mask on the metalized layer exposing a portion of the biocompatible substrate layer in accordance with one embodiment of the present.

FIG. 3E is an elevation drawing illustrating etching of a portion of the biocompatible substrate and removal of the second Photoresist mask in accordance with one embodiment of the present.

FIG. 3F is an elevation drawing illustrating bonding of a device component to metalized contacts around a fenestration in the biocompatible substrate layer with anisotropic conductive adhesive (ACA) in accordance with one embodiment of the present.

### DETAILED DESCRIPTION

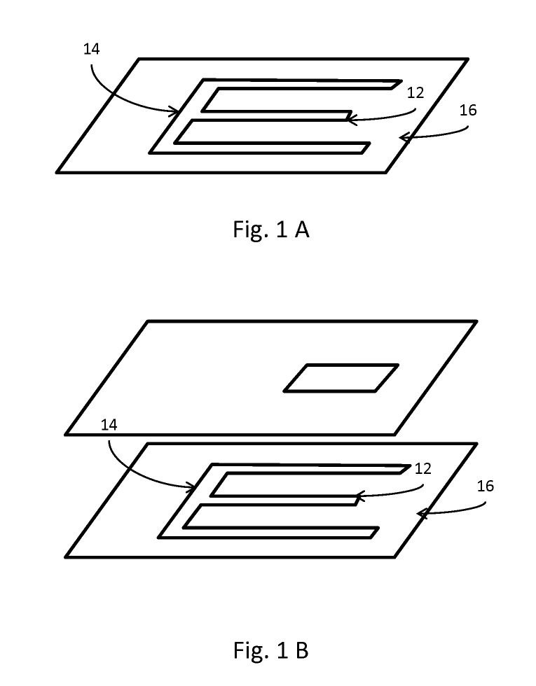

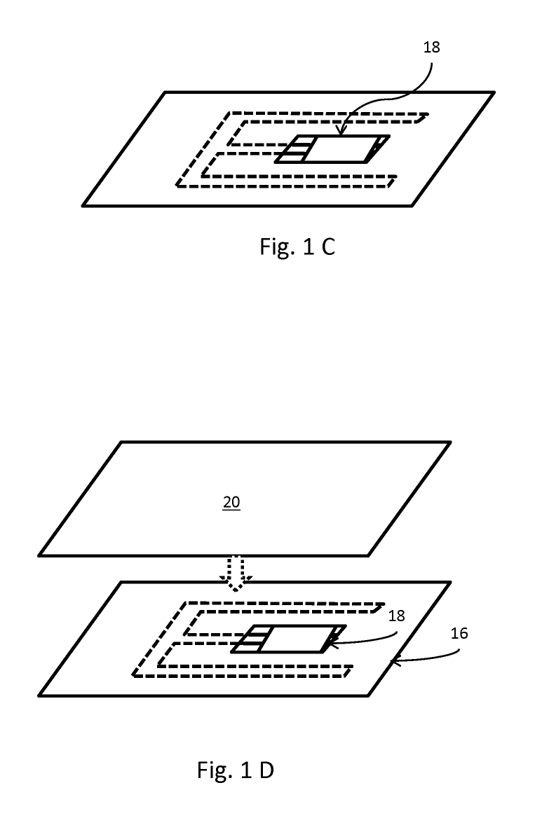

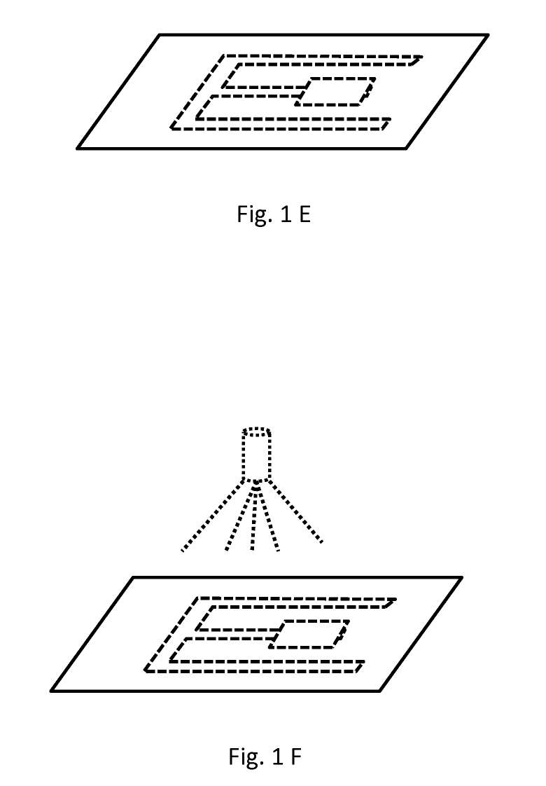

In one embodiment of the present invention, a method for manufacturing a biocompatible and radio transparent implantable sensor package. Such a method is illustrated in FIG. 1A-1F. Metal pads 12 and antenna 14 are disposed on a liquid crystal polymer (LCP) sheet 16. In one embodiment, this sheet is configured in a rectangular shape, while one skilled in the art will appreciate that other shapes may be used depending on design choices. The sheet 16 may be disposed upon a flat mold plate (not shown). Electronics 18 may be disposed upon the layer of liquid crystal polymer sheet 16. A second layer of liquid crystal polymer (LCP) material 20 is then be draped over the first 16. There are two ways that the LCP can be sealed. First, a second mold half, having at last one cavity, is positioned over the second layer of liquid crystal polymer 20. The mold halves may then be compressed, thereby adhering opposing surfaces of the two liquid crystal polymer layers together, thereby sealing the electronics, metal pads, and antenna components between the sheets. Second, the use of a laser to heat the outline of the package may be used to adhere the two layers of LCP. In alternative embodiments, additional layers may be disposed between the layers, having suitable excisions to build up the area surrounding the electronics package if so desired. Once the layers are adhered, the package may be cut to desired shapes and any desired external coating may be applied.

One embodiment of the present invention provides packaging able to be constructed and allow internal components to interact with the environment while still retaining hermeticity. This incorporates the construction of a “window” into the LCP packaging and using the ACA to connect and create a hermetic seal for the component needing to interact with the environment.

In one embodiment of the present invention LCP is used as a material that provides near hermetic seals. Studies have determined that when LCP works in liquid phase it remains a hermetic seal. Implantation in the body will therefore allow LCP to be hermetic in the system. One skilled in the art will appreciate that other materials may be used that exhibit suitable properties, including radio transparency and high hermeticity.

In one embodiment of the present invention, the package may be configured in a tadpole design, with a long tensioning ring section for fixation in a biological space as well as for housing the metal antenna traces. Such a configuration facilitates implantation via syringe type inserter. One skilled in the art will appreciate that such a configuration is facilitated by the method according to FIGS. 1A-1F. Such a configuration has many advantages, including but not limited to decreases in sharp edges and burrs which might result in tissue necrosis or fibrosis, sub micron scale miniaturization, and other

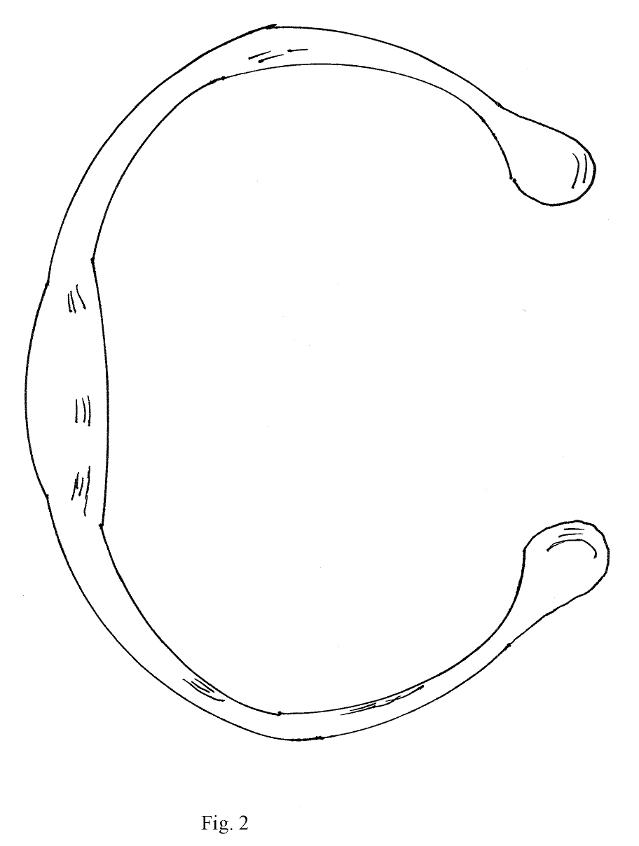

One embodiment of the present invention provides a package for the implantation of an intraocular pressure sensor as illustrated in FIG. 2. In such an embodiment, the sensor package is provided with a crescent shaped or demi-annular configuration, although one skilled in the art will appreciate that other embodiments of the present invention may employ other shapes depending on the location, design, and method for implantation. The package is configured to be transparent to radio waves and small in size. At first and second termini and at a point of the arc between those termini, bulbous projections within the plane of the structure are provided for housing electronics and sensor equipment. Such a configuration is suited to minimally invasive implantation. In an alternative embodiment, a large projection exists at the first termini for containing electronics and sensor components, with a minor bulbous projection at the second termini. In an additional embodiment, the implant may be oblong in shape and configured to be implanted surgically.

The package is configured from biocompatible material having high radio transparency. Materials used in the manufacture of the package include low temperature co-fired ceramic, liquid crystal polymer, Poly(methyl methacrylate) or other silicon based materials. One skilled in the art will appreciate that the properties of these materials lend themselves to different applications, but while each material exhibits properties suitable for specific implantable medical applications, all are radio transparent and provide a format for small scale implantable devices.

In order to expose the capacitive sensor to the external environment, a window is constructed into the LCP. With a window created in the LCP, the capacitive sensor is able to interact with its surrounding, while the powering, transmitting, and conversion circuitry is sealed from the biology.

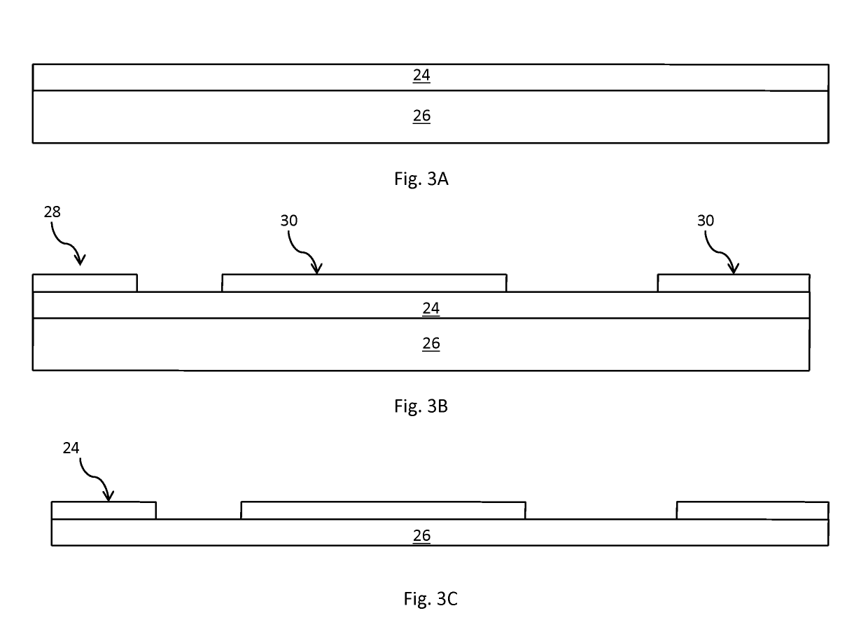

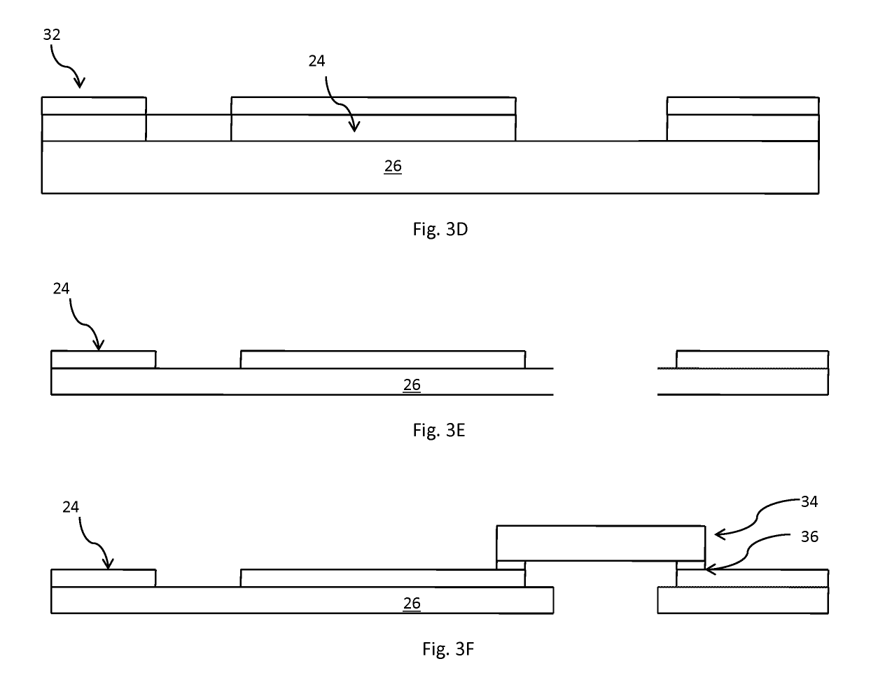

Any measurement component may be incorporated into the device to interact with the external environment. In one embodiment, a means of providing interaction with the external environment is to expose a MEMS capacitor or other such device component to the environment. Steps for introducing such a fenestration are describe with reference to FIGS. 3A-3F. The first step is a mask is created that outlines the shape of the window for the sensor. In one embodiment a MEMS capacitor is use obtained from Microfab Bremen, the windows dimensions are 760 microns in length and 600 microns in width. One skilled in the art will appreciate that other embodiments may employ other devised and the window/fenestration six would be dependent on the device and application. Using photolithography of the window mask after copper traces have been fabricated on the LCP, photoresist is applied to the LCP to outline the window for deep reactive ion etch (DRIE). The DRIE method etches away LCP creating the necessary window. Once the LCP processing is completed, the MEMS sensor is attached to the correct traces, placed over the window, and connected using anisotropic conductive adhesive (ACA). Finally, in one such embodiment the LCP, ACA, and sensor are heated in a magnetic oven to create conductivity between the MEMS sensor and the traces, completing the fabrication and sealing the LCP and sensor, so that there is no leakage at the window.

One embodiment of the present invention comprises the use of low temperature co-fired ceramic or liquid crystal polymer device as the package body. Such an embodiment is illustrated in FIGS. 3A-3F. In such an embodiment, the layers of ceramic material are built up as they would in a non-implantable telecommunication device. In such an embodiment, a metalized layer 24 may be disposed on a LCP substrate 26. Photoresist 28 is applied to the metalized layer 24 to define a pattern of traces 30 on the metalized layer 24. The metalized layer 24 is etched and the photoresist is removed.

For applications where fenestrations, apertures, or windows may be desirable due to the nature or function of the electronics to be disposed in the package, additional processing steps may be desired. As illustrated in FIG. 3D-3F an additional layer of photoresist 32 may be applied to the metalized layer 24 and to exposed areas of the substrate 26. A fenestration, window or aperture 34 is made in the substrate 26 by deep reactive ion etching (DRIE) of the exposed substrate 26. An electrical component 32 may then be disposed across the fenestration 34, and coupled to said metalized layer 24 with anisotropic conductive adhesive 36.

One skilled in the art will appreciate that materials for implantation into the human body be tested for biocompatibility as well as efficacy in their selected role. Low temperature co-fired ceramic and liquid crystal polymer devices are provided for integration into an implantable electronic device. Package materials can be selected from the range provided based on biocompatibility score in the intended environment.

In one embodiment the devices are configured to be 6 mm by 3 mm for insertion. In one such embodiment, the selected low temperature co-fired ceramic is selected from the group of low temperature co-fired ceramics consisting of DuPont 951 green tape; DuPont 943 green tape, and Heraeus HL2000 green tape. The materials thus selected and formed into the desired package configuration are then fired. These fired packages were tested for biocompatibility. In one embodiment the testing process comprises first preconditioning the green tape materials at 120 degrees Celsius for 30 minutes. Once the materials are pre-conditioned, they are punched using a Unichem MP4150M punch machine or equivalent, to give us the dimensions that we are testing. After punching, layers are combined together using a lamination and pressing technique in an iso-static press to form a desired shape. After pressing, the material is then cut into the predetermined shape. Finally, the material is put into an oven where temperatures are ramped up to 800 degrees Celsius over an eight hour period. The remaining organics are burned out, the material hardens, becoming ceramic. This final product is then tested for biocompatibility.

In alternative embodiments, materials other than liquid crystal polymer (LCP) are used. In some embodiments, low temperature co-fired ceramics (LTCC), silicon, anisotropic conductive adhesive (ACA), poly(methyl methacrylate), parlyene, and alumina are used.

The foregoing description of the embodiments of the invention has been presented for the purposes of illustration and description. It is not intended to be exhaustive or to limit the invention to the precise form disclosed. Many modifications and variations are possible in light of this disclosure. It is intended that the scope of the invention be limited not by this detailed description, but rather by the claims appended hereto.

We claim:

1. A system for the packaging of wireless electronics and sensors in a mammalian body, the system comprising:
a package configured in a shape suitable for implantation in said body;
said package being configured from a biocompatible material; and
said biocompatible material having a high degree of radio wave transparency.

2. The system of claim 1 wherein said biocompatible material is selected from the group of biocompatible materials consisting of liquid crystal polymer, poly(methyl methacrylate), low temperature co-fired ceramic, anisotropic conductive adhesive, silicon and combinations of thereof.

3. The system according to claim 1 wherein said package is provided with a plurality of layers of said biocompatible material, said layers being applied in a pattern, said pattern defines a structure in said shape suitable for implementation in said body.

4. The system according to claim 1 wherein said high degree of radio wave transparency is a degree of transparency that allows signals transmitted from an antenna disposed within said package to be transmitted to a receiver disposed externally to said body at a distance of about approximately 2 meters.

5. The system according to claim 1 wherein said high degree of radio wave transparency comprises a loss of not greater than 50 dB from an antenna disposed within a mammalian body.

6. The system according to claim 4 wherein said high degree of radio wave transparency comprises having not greater than 50 dB of loss from an antenna disposed within said package.

7. The system according to claim 1 further comprising a biocompatible coating.

8. The system according to claim 7 wherein said biocompatible coating comprises parylene.

9. A method for the manufacture of an implantable electronics package, said method comprising:
Placing a first biocompatible material sheet on a first mold plate;
Patterning an antenna and metal contacts upon a top surface of said first biocompatible material sheet;
Disposing an electronics package upon said metal contacts;
Draping a second sheet of biocompatible material over said first biocompatible material sheet and said electronics package;
Compressing said second sheet of biocompatible material against said top surface with a second mold plate having a recess for receiving said electronics package.

10. The method according to claim 9 wherein said biocompatible material is a biocompatible selected from the group of materials consisting of liquid crystal polymer, poly(methyl methacrylate), low temperature co-fired ceramic, anisotropic conductive adhesive, silicon and combinations of thereof.

11. The method according to claim 9, said method further comprising: creating a fenestration cut into said first biocompatible material sheet, thereby allowing internal components to interact with an environment external to said package.

12. The method according to claim 11 wherein said creating said fenestration comprises deep reactive ion etching said first biocompatible material sheet forming said fenestration.

13. The method according to claim 11 further comprising hermetically sealing said first biocompatible material sheet proximate to said fenestration with Anisotropic conductive adhesive to least one component of said electronics package, said component being disposed proximate to said fenestration, such that at least a portion of said component is exposed to said environment external to said implantable package.

14. A method for creation of a hermetic seal in an implantable package, said method comprising:
Placing a first biocompatible material sheet on a first mold plate;
Patterning an antenna and metal contacts upon a top surface of said first biocompatible material sheet;
Disposing an electronics package upon said metal contacts;
Draping a second sheet of biocompatible material over said first biocompatible material sheet and said electronics package;
laser sealing said second sheet of biocompatible material to said top surface with a second mold plate having a recess for receiving said electronics package.

15. The method according to claim 14, said method further comprising: creating a fenestration cut into said first biocompatible material sheet, thereby allowing internal components to interact with an environment external to said package.

16. The method according to claim 15 wherein said creating said fenestration comprises deep reactive ion etching said first biocompatible material sheet forming said fenestration.

17. The method according to claim 15 further comprising hermetically sealing said fenestration with Anisotropic conductive adhesive to least one component of said electronics package, said component being disposed proximate to said fenestration, such that said component is exposed to said environment external to said implantable package.

18. The method according to claim 14, wherein said first and second biocompatible material sheets are each comprised of a biocompatible material selected from the group of biocompatible materials consisting of liquid crystal polymer, poly(methyl methacrylate), low temperature co-fired ceramic, anisotropic conductive adhesive, silicon and combinations of thereof.
